(19) United States
(12) Patent Application Publication
(10) Pub. No.: US 2024/0315533 A1
(43) Pub. Date: Sep. 26, 2024

(54) REMOTE USER INTERFACE FOR AN ENDOSCOPE

(71) Applicant: Boston Scientific Scimed Inc., Maple Grove, MN (US)

(72) Inventors: Ryan V. Wales, Northborough, MA (US); Paul Smith, Smithfield, RI (US); Barry Weitzner, Acton, MA (US); Gary A. Jordan, Litchfield, NH (US); Scott E. Brechbiel, Burlington, MA (US); Kurt Nicholas Robakiewicz, Upton, MA (US); John B. Golden, Norton, MA (US); Narunn Suon, Lawrence, MA (US)

(73) Assignee: Boston Scientific Scimed Inc., Maple Grove, MN (US)

(21) Appl. No.: 18/613,707

(22) Filed: Mar. 22, 2024

Related U.S. Application Data
(60) Provisional application No. 63/454,359, filed on Mar. 24, 2023.

(51) Int. Cl. A61B 1/00; A61B 1/015

(52) U.S. Cl. CPC A61B 1/00068; A61B 1/00016; A61B 1/00042; A61B 1/015

(57) ABSTRACT

An example medical device includes an endoscope. The endoscope includes a handle including a central control section, one or more valves coupled to the central control section, an elongate shaft extending distally from the handle to a distal tip, wherein the elongate shaft is configured to be inserted into a body cavity, and a remote user interface operatively coupled to the central control section. The remote user interface is configured to operate the one or more valves to control a flow of fluid through the elongate shaft.

### CROSS-REFERENCE TO RELATED APPLICATIONS

This application claims the benefit of U.S. Provisional Patent Application Ser. No. 63/454,359 filed on Mar. 24, 2023, the disclosure of which is incorporated herein by reference.

### TECHNICAL FIELD

The present disclosure relates generally to the field of medical devices. More particularly, the present disclosure relates to devices, systems and methods for remotely operating an endoscope or steerable catheter.

### BACKGROUND

Medical devices, such as steerable/deflectable endoscopes and/or catheters, may be used to perform various diagnostic and/or treatment procedures. For example, a physician may perform aggressive interventional and therapeutic endoscopic procedures, such as, for example, full thickness removal of large tissue lesions, tunneling under the mucosal layer of the gastrointestinal tract or respiratory system, repair of post-surgical complications, thoracic surgery, and/or pleural space procedures. The various procedures may require different devices and/or different physical actions by the practitioner. Endoscopes provide working channels for a plurality of individually articulating tools, however, articulating each of the individual tools tends to be ergonomically difficult to control by a single physician.

Further, to perform such interventional and therapeutic procedures, the physician may need to perform a lens wash, irrigation, and/or aspiration of debris and fluids. Conventionally, the physician and/or user utilizes an air/water valve and a suction valve, which are positioned on a handle of the endoscope, by depressing the valves using their finger. Managing the valves as well as individually articulating tools by a single user tends to be difficult. It is with these considerations that the improvements of the present disclosure may be useful.

Of the known medical devices, systems, and methods, each has certain advantages and disadvantages. There is an ongoing need to provide alternative medical devices and systems, including controls for manipulating and maneuvering such medical devices.

### BRIEF SUMMARY

This disclosure provides design, material, manufacturing method, and use alternatives for medical devices. An example medical device may include an endoscope. The endoscope may include a handle including a central control section, one or more valves coupled to the central control section, an elongate shaft may extend distally from the handle to a distal tip, wherein the elongate shaft may be configured to be inserted into a body cavity, and a remote user interface operatively coupled to the central control section, the remote user interface may be configured to operate the one or more valves to control a flow of fluid through the elongate shaft.

Alternatively or additionally to any of the embodiments above, the remote user interface may include one or more buttons configured to be actuated to operate the one or more valves.

Alternatively or additionally to any of the embodiments above, the one or more buttons may be attached to one or more flexible tethers, the one or more flexible tethers configured to engage with the one or more valves.

Alternatively or additionally to any of the embodiments above, the remote user interface may include one or more flexible tethers coupled to a foot pedal, the one or more flexible tethers configured to engage with the one or more valves.

Alternatively or additionally to any of the embodiments above, the remote user interface may include an actuator box coupled to the central control section of the handle, the actuator box positioned over the one or more valves.

Alternatively or additionally to any of the embodiments above, the remote user interface may further include a wireless remote configured to wirelessly communicate with a receiver positioned within the actuator box.

Alternatively or additionally to any of the embodiments above, the actuator box may include one or more fixed magnets and one or more floating magnets, wherein the wireless remote is configured to control a polarity of the one or more fixed magnets such that the one or more fixed magnets may include a first polarity in which the one or more fixed magnets attract the one or more floating magnets to a first position, and the one or more fixed magnets may include a second polarity in which the one or more fixed magnets repel the one or more floating magnets to a second position, the one or more floating magnets configured to engage with the one or more valves when in the second position.

Alternatively or additionally to any of the embodiments above, the actuator box may include one or more actuator buttons configured to engage with the one or more valves, wherein the wireless remote is configured to control the one or more actuator buttons.

Alternatively or additionally to any of the embodiments above, the remote user interface may include one or more flexible tethers coupled to one or more syringes, the one or more flexible tethers configured to engage with the one or more valves and the one or more syringes configured to advance a fluid distally to control the flow of fluid through the elongate shaft.

Another example medical device may include a handle including a central control section, one or more valves coupled to the central control section, the one or more valves may include one or more valves, an elongate shaft extending distally from the handle to a distal tip, the elongate shaft configured to be inserted into a body cavity, and a remote user interface operatively coupled to the central control section. The remote user interface may be configured to operate the one or more valves to control a flow of fluid through the elongate shaft, wherein the remote user interface may include one or more buttons configured to be actuated to operate the one or more valves.

Alternatively or additionally to any of the embodiments above, the one or more valves may be removed from one or more valve wells, and replaced with one or more tethers, the one or more tethers coupled to the remote user interface.

Alternatively or additionally to any of the embodiments above, the remote user interface includes an actuator box coupled to the central control section of the handle, the actuator box may be positioned over the one or more valves.

Alternatively or additionally to any of the embodiments above, the remote user interface may further include a wireless remote configured to wirelessly communicate with a receiver positioned within the actuator box.

Alternatively or additionally to any of the embodiments above, the remote user interface may further include a wireless remote configured to wirelessly communicate with a receiver positioned at the control section of the handle.

Alternatively or additionally to any of the embodiments above, the actuator box may include one or more fixed magnets and one or more floating magnets, wherein the wireless remote may be configured to control a polarity of the one or more fixed magnets such that the one or more fixed magnets include a first polarity in which the one or more fixed magnets attract the one or more floating magnets to a first position, and the one or more fixed magnets include a second polarity in which the one or more fixed magnets repel the one or more floating magnets to a second position, the one or more floating magnets configured to engage with the one or more valves when in the second position.

Another example medical device may include an endoscope including a handle including a central control section, one or more valves coupled to the central control section, a receiver positioned adjacent the control section of the handle, an elongate shaft extending distally from the handle to a distal tip, the elongate shaft configured to be inserted into a body cavity, and a remote user interface operatively coupled to the central control section. The remote user interface may be configured to operate the one or more valves to control a flow of fluid through the elongate shaft. The remote user interface may include a wireless remote configured to wirelessly communicate with the receiver.

Alternatively or additionally to any of the embodiments above, the receiver may be positioned within an actuator box, the actuator box coupled to the handle.

Alternatively or additionally to any of the embodiments above, the actuator box may include one or more buttons configured to engage with the one or more valves.

Alternatively or additionally to any of the embodiments above, the remote user interface may include one or more sensors configured to communicate with the receiver.

Alternatively or additionally to any of the embodiments above, the actuator box may include one or more fixed magnets and one or more floating magnets, wherein the wireless remote may be configured to control a polarity of the one or more fixed magnets such that the one or more fixed magnets include a first polarity in which the one or more fixed magnets attract the one or more floating magnets to a first position, and the one or more fixed magnets may include a second polarity in which the one or more fixed magnets repel the one or more floating magnets to a second position, the one or more floating magnets configured to engage with the one or more valves when in the second position.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates example components of an endoscope;

FIG. 2 depicts components of an endoscope system with endoscope, light source, light source connector, water reservoir, and tubing assembly for air and lens wash fluid delivery;

FIG. 3 illustrates exemplary tethers for a remote user interface;

FIG. 4 illustrates the exemplary tethers, as in FIG. 3 connected to a central control section of an endoscope, and the remote user interface;

FIG. 5 illustrates exemplary tethers connected to a central control section of an endoscope, and a remote user interface;

FIG. 6 illustrates an exemplary remote user interface having a first actuator and a second actuator;

FIG. 7 illustrates an exemplary remote user interface;

FIG. 8A illustrates an exemplary central control section of an endoscope and an exemplary actuator box;

FIG. 8B illustrates the exemplary central control section of the endoscope and the exemplary actuator box of FIG. 8A, attached to the central control section;

FIG. 9 illustrates an exemplary central control section of an endoscope and an exemplary actuator box;

FIG. 10A illustrates an exemplary central control section of an endoscope including a first actuator button and a second actuator button;

FIG. 10B illustrates a side view of the first actuator button, as in FIG. 10A;

FIG. 10C illustrates a perspective view of the first actuator button, as in FIG. 10A;

FIG. 10D illustrates a perspective view of the second actuator button, as in FIG. 10A; and

FIG. 11 illustrates a side view of an exemplary actuator button.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

For the following defined terms, these definitions shall be applied, unless a different definition is given in the claims or elsewhere in this specification. All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure. The recitation of numerical ranges by endpoints includes all numbers within that range (e.g., 1 to 5 includes, 1, 1.5, 2, 2.75, 3, 3.80, 4, and 5).

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the claims.

Conventionally, endoscope devices may be used to perform various diagnostic and/or treatment procedures. For example, a physician may perform aggressive interventional and therapeutic endoscopic procedures, such as, for example, full thickness removal of large tissue lesions, tunneling under the mucosal layer of the gastrointestinal tract or respiratory system, repair of post-surgical complications, thoracic surgery, and/or pleural space procedures. The various procedures may require different devices and/or different physical actions by the practitioner. Endoscopes provide working channels for a plurality of individually articulating tools, however, articulating each of the individual tools tends to be ergonomically difficult to control by a single physician.

Further, to perform such interventional and therapeutic procedures, the physician may need to perform a lens wash, irrigation, and/or aspiration of debris and fluids. Conventionally, the physician and/or user utilizes an air/water valve and a suction valve, which are positioned on a handle of the endoscope, by depressing the valves using their finger. Managing the valves as well as individually articulating tools by a single user tends to be difficult. Disclosed herein are methods and systems to reduce or eliminate the challenges of controlling the individual tools by a single physician.

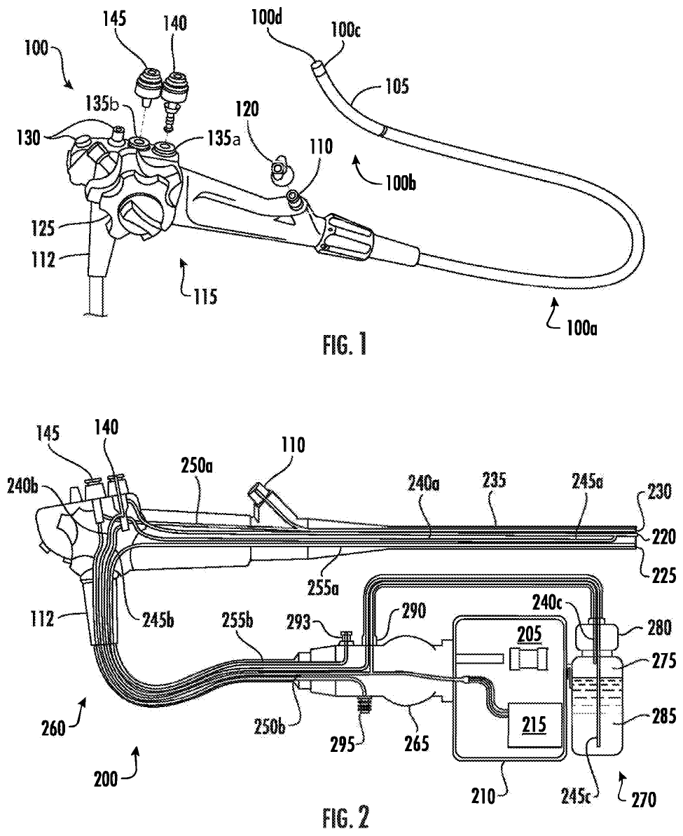

With reference to FIGS. 1-2, an exemplary endoscope 100 and system 200 are depicted that may comprise an elongated shaft 100a coupled to a handle 112. The elongated shaft 100a may extend distally from the handle 112 to a distal tip 100c that may be configured to be inserted into a body cavity of a patient. The handle may include a central control section 115. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The elongated shaft 100a may include the distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to the central control section 115 of the endoscope 100. A biopsy valve 120 may be utilized to seal the channel opening 110 against unwanted fluid egress.

The central control section 115 may be provided with knobs 125 for providing remote 4-way steering of the distal tip via wires connected to the articulation joint in the flexible bending portion 105 (e.g., one knob controls up-down steering and another knob control for left-right steering). A plurality of video switches 130 for remotely operating the video processing unit 210 may be arranged on a proximal end side of the central control section 115. In addition, the central control section 115 is provided with a first valve well 135a and a second valve well 135b. The first valve well 135a may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The second valve well 135b receives a suction valve 145 for operating a suction operation. A suction supply line 250a runs distally from the suction valve 145 along the shaft 100a to a junction point in fluid communication with the working channel 235 of the endoscope 100.

The central control section 115 is electrically and fluidly connected to the video processing unit 210, via a flexible umbilical 260 and connector portion 265 extending therebetween. The flexible umbilical 260 has a gas (e.g., air or CO2) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the elongated shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the first valve well 135a for the gas/water valve 140 and configured such that operation of the gas/water valve in the well controls supply of gas or lens wash to the distal tip 100c of the endoscope 100. The suction feed line 250b is fluidly connected to the second valve well 135b for the suction valve 145 and configured such that operation of the suction valve 145 in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connection portion 265 and branched to the gas/water valve 140 on the central control section 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

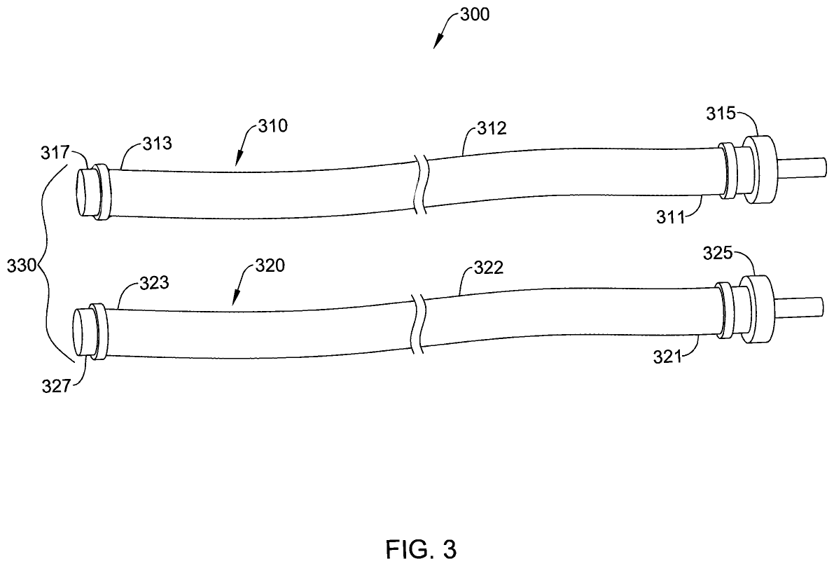

FIG. 3 illustrates exemplary tethers 300 for a remote user interface 330. As shown in FIG. 2, the tethers 300 may include a first tether 310 and a second tether 320. In some cases, it may be contemplated that the tethers 300 may include only one tether formed from a coextrusion, and the one tether may include a dual interface configured to engage with the first and second valve wells 135a, 135b, respectively. The one tether may include a singular actuator button at a proximal end configured to control the functions of both the first and second valve wells 135a, 135b, respectively. The tethers 300 may include a length in a range of about 1 m (meter) to about 4 m, or any other suitable length, as desired. In some cases, the tethers may include an outer diameter in a range of about 8 mm (millimeters) to about 14 mm, or any other suitable outer diameter, as desired. In some cases, the tethers 300 may be considered to be flexible tethers and may be formed from a braid. In some cases, the tethers 300 may be formed from a polymer such as polyethylene, polypropylene, polyurethane, or any other suitable material, as desired.

The first tether 310 may include an elongate tube 312 having a distal end 311 and a proximal end 313. The first tether 310 may include a first interface 315 positioned at the distal end 311 of the elongate tube 312. The first interface 315 may be configured to engage with a valve well (e.g., first valve well 135a) on an endoscope. A first actuator button 317 may be positioned at the proximal end 313. The first actuator button 317 may be configured to be depressed, which may actuate a strain within the elongate tube 312 (although not explicitly shown). The strain may advance distally to engage with the valve well to control a flow of fluid through the elongate shaft (e.g. elongated shaft 100a). The first actuator button 317 may include a first position and a second position, in which a user may control the gas/water functions of the valve 140. For example, the first actuator button 317 may be depressed to a first position, in which a vent is blocked to the atmosphere. Gas is allowed to flow from the first valve well 135a down the gas supply line 240a and out the distal tip 100c of the endoscope 100 in order to, for example, insufflate the treatment area of the patient. When the first actuator button 317 is pressed downward to a second position, gas is blocked from exiting the valve, allowing pressure of the air passing from an air pump to rise in a water reservoir. Pressurizing the water source forces water out of a lens wash tubing, through a connector portion, umbilical, through the first valve well 135a and down a lens wash supply line, converging with a gas supply line prior to exiting a distal tip of the endoscope 100 via a gas/lens wash nozzle. In some cases, the fluid may be water to be used for a lens wash, for example. In some cases, the fluid may be a gas (e.g., air) for insufflation, for example.

The second tether 320 may include an elongate tube 322 having a distal end 321 and a proximal end 323. The second tether 320 may include a second interface 325 positioned at the distal end 321 of the elongate tube 322. The second interface 325 may be configured to engage with a valve well (e.g., second valve well 135b) on an endoscope. A second actuator button 327 may be positioned at the proximal end 323 of the elongate tube 322. The second actuator button 327 may be configured to be depressed, which may actuate a strain within the elongate tube 322 (although not explicitly shown). The strain may advance distally to engage with the second valve well 135b to control a suction of fluid through the elongate shaft (e.g. elongated shaft 100a), in a manner similar to that described above with reference to FIGS. 1 to 2.

The tethers 300 may be configured to engage with the valve wells (e.g., valve wells 135a, 135b) via the first and second interfaces 315, 325 and extend the function of the valves 140, 145 via the elongate tubes 312, 322, such that a second user may control the gas/water valve 140 and the suction valve 145 using the remote user interface 330, e.g., the first and second actuator buttons 317, 327, respectively. In use, for example, while a first user is manipulating the endoscope and the various articulating tools, the second user may control (e.g., actuate) the gas/water and suction valve functions via a remote location. While it is discussed that the tethers 300 may be used with an endoscope (e.g., endoscope 100), for example, it may be contemplated that the tethers 300 may be used with a colonoscope, a gastroscope, a bronchoscope, a ureteroscope, or any other suitable scope.

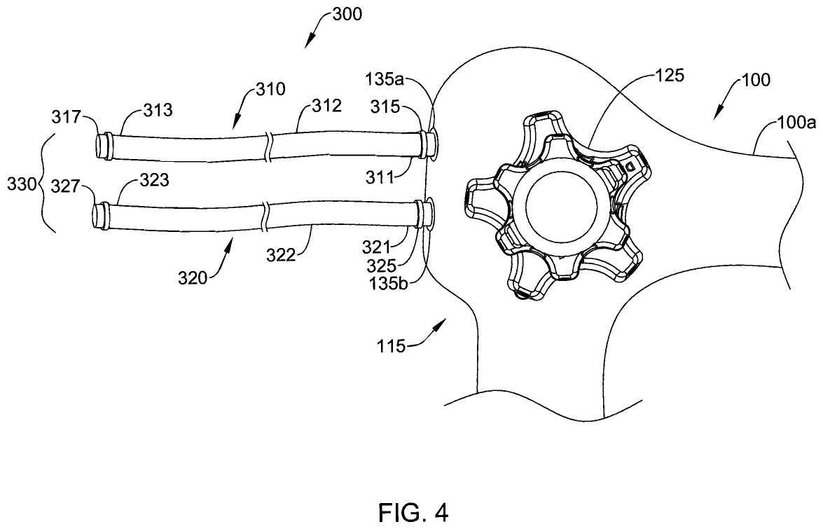

FIG. 4 illustrates the exemplary tethers 300, as in FIG. 3 connected to the central control section 115 of the endoscope 100, and the remote user interface 330. As discussed with reference to FIG. 3, the tethers 300 may include the first tether 310 and the second tether 320. The first interface 315 of the first tether 310 may be coupled to the first valve well 135a. The first interface 315 may include a size and shape so as to provide a seal between the first interface 315 and the first valve well 135a. The first interface 315 may be coupled to the first valve well 135a via an interference fit, a snap fit, or any other suitable type of fit, as desired.

The first actuator button 317 may be positioned at the proximal end 313 of the elongate tube 312. The first actuator button 317 may be configured to be depressed, which may actuate a strain within the elongate tube 312 (although not explicitly shown). The strain may advance distally to engage with the valve well to control a flow of fluid through the elongate shaft (e.g. elongated shaft 100a). In some cases, the fluid may be water to be used for a lens wash, for example. In some cases, the fluid may be a gas (e.g., air) for insufflation, for example.

The second tether 320 may include the elongate tube 322 having a distal end 321 and a proximal end 323. The second tether 320 may include the second interface 325 positioned at the distal end 321 of the elongate tube 322. The second interface 325 may be configured to engage with a valve well (e.g., second valve well 135b) on an endoscope. The second interface 325 may include a size and shape so as to provide a seal between the second interface 325 and the second valve well 135b. The second interface 325 may be coupled to the second valve well 135b via an interference fit, a snap fit, or any other suitable type of fit, as desired. A second actuator button 327 may be positioned at the proximal end 323. The second actuator button 327 may be configured to be depressed, which may actuate a strain within the elongate tube 322 (although not explicitly shown). The strain may advance distally to engage with the valve well to control a suction of fluid through the elongate shaft (e.g. elongated shaft 100a).

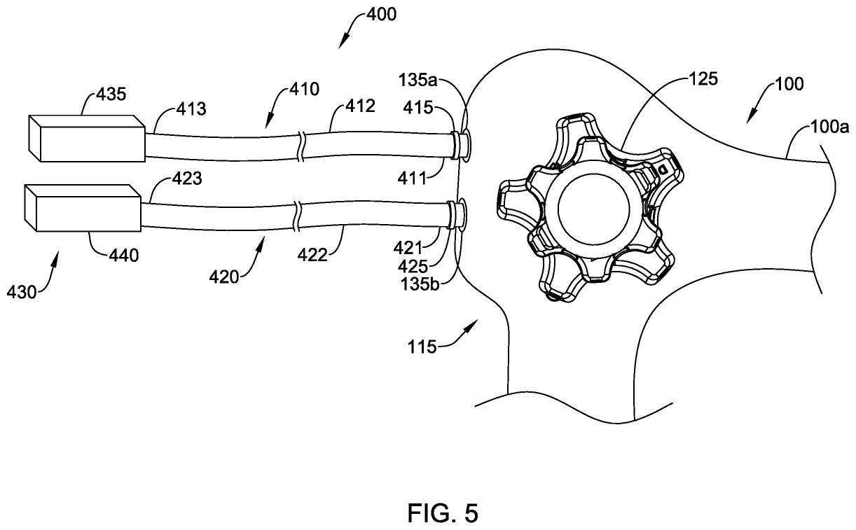

FIG. 5 illustrates exemplary tethers 400 connected to the central control section 115 of the endoscope 100, and a remote user interface 430. The tethers 400 may include a first tether 410 and a second tether 420. The first tether 410 may include an elongate tube 412 having a distal end 411 and a proximal end 413. A first interface 415 may be positioned at the distal end 411 of the first tether 410. The first interface 415 of the first tether 410 may be coupled to the first valve well 135a. The first interface 415 may include a size and shape so as to provide a seal between the first interface 415 and the first valve well 135a. The first interface 415 may be coupled to the first valve well 135a via an interference fit, a snap fit, or any other suitable type of fit, as desired.

The second tether 420 may include an elongate tube 422 having a distal end 421 and a proximal end 423. The second tether 420 may include a second interface 425 positioned at the distal end 421 of the elongate tube 422. The second interface 425 may be configured to engage with the second valve well 135b. The second interface 425 may include a size and shape so as to provide a seal between the first second interface 425 and the second valve well 135b. The second interface 425 may be coupled to the second valve well 135b via an interference fit, a snap fit, or any other suitable type of fit, as desired.

The remote user interface 430 may include a first foot pedal 435 and a second foot pedal 440. The first foot pedal 435 may be coupled to the proximal end 413 of the elongate tube 412 of the first tether 410. The second foot pedal 440 may be coupled to the proximal end 423 of the elongate tube 422 of the second tether 420. The first foot pedal 435 may be configured to be actuated by a user's foot which may actuate a strain within the elongate tube 412 (although not explicitly shown). For example, the user may depress the pedal (e.g., by stepping on the pedal) which may actuate the strain in a distal direction, and the strain may engage with the first valve well 135a to control a flow of fluid through the elongate shaft (e.g. elongated shaft 100a). In some cases, the fluid may be water to be used for a lens wash, for example. In some cases, the fluid may be a gas (e.g., air) for insufflation, for example.

In some cases, the second food pedal 440 may be configured to be actuated by a user's foot which may actuate a strain within the elongate tube 422 (although not explicitly shown. For example, the user may depress the pedal (e.g., by stepping on the pedal) which may actuate the strain in a distal direction, and the strain may engage with the second valve well 135b to control a suction of fluid through the elongate shaft (e.g. elongated shaft 100a).

While the remote user interface 430 is illustrated as being two separate pedals (e.g., the first foot pedal 435 and the second foot pedal 440), it may be contemplated the remote user interface 430 may be a singular foot pedal and may be configured to be actuated by a user's foot, which may actuate one or more strains within a singular elongate tube. In some cases, when there is a singular foot pedal, the elongate tube may include a bifurcated distal end which may include the first interface 415 and the second interface 425. When a singular foot pedal is used, a user may depress a first side of the foot pedal to actuate a first strain, and/or may depress a second side of the foot pedal to actuate a second strain. This is just an example.

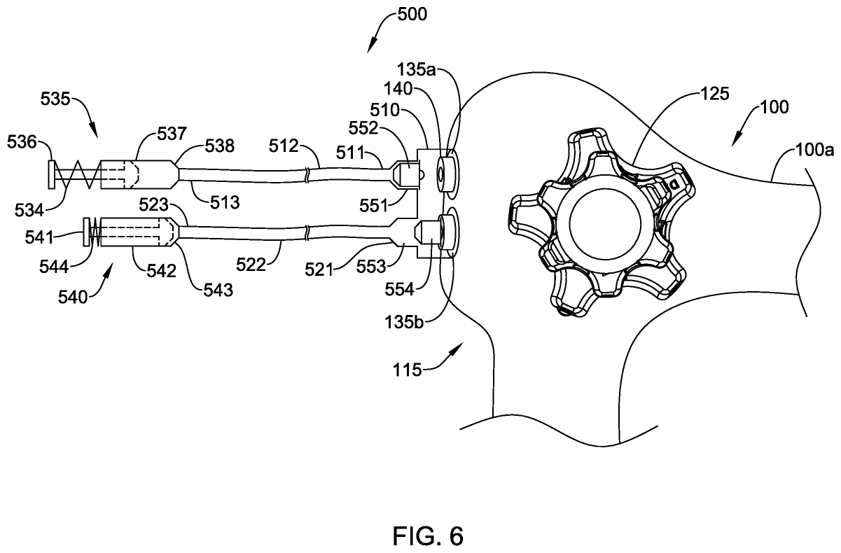

FIG. 6 illustrates an exemplary remote user interface 500 having a first actuator 535 and a second actuator 540. The remote user interface 500 may include one or more syringes configured to administer a fluid (e.g., air, water, saline, or the like) through a tubing to an actuator box 510. In some cases, the remote user interface 500 may include the first actuator 535, which may include a first barrel 537, a first plunger 536, and a first tip 538. The first plunger 536 may include a spring 534 that may be configured to bias the first plunger 536 to a non-deployed position (as illustrated by the first plunger 536). The first tip 538 may be coupled to a proximal end 513 of a first tube 512. The first tube 512 may include a distal end 511 that may be coupled to a first fluid chamber 551 of the actuator box 550. The first tube 512 may include a lumen extending from the proximal end 513 to the distal end 511, although this is not explicitly shown. The first fluid chamber 551 may be in fluid communication with the lumen of the first tube 512. In some cases, a first actuator button 552 may be positioned within the first fluid chamber 551, and may be configured to move distally within the actuator box 510 and engage with the gas/water valve 140, to control a flow of fluid through the elongated shaft 100a.

In some cases, the remote user interface 500 may include the second actuator 540, which may include a second barrel 542, a second plunger 541, and a second tip 543. The second plunger 541 may include a spring 544 that may be configured to bias the second plunger 541 to a non-deployed position (as illustrated by the first plunger 536). In some cases, when the first plunger 536, and/or the second plunger 541 is depressed (e.g., actuated) in a distal direction, the spring(s) 534, 544 compresses (as illustrated by the second plunger 541). The second tip 543 may be coupled to a proximal end 523 of a second tube 522. The second tube 522 may include a distal end 521 that may be coupled to a second fluid chamber 553 of the actuator box 510. The second tube 522 may include a lumen extending from the proximal end 523 to the distal end 521, although this is not explicitly shown. The second fluid chamber 553 may be in fluid communication with the lumen of the second tube 522. In some cases, a second actuator button 554 may be positioned within the second fluid chamber 553, and may be configured to move distally within the actuator box 550 and engage with the suction valve 145 (as illustrated by the second actuator button 554), to control a suction through the elongated shaft 100a.

In some cases, a user may actuate the first plunger 536 by pressing the first plunger 536 in a distal direction toward the first barrel 537 (as illustrated by the second plunger 541), and the first plunger 536 forces a fluid (e.g., gas, water, saline, or the like) through the first tip 538 and the lumen of the first tube 512 into the first fluid chamber 551. The fluid creates pressure within the first fluid chamber 551, and the pressure then forces the first actuator button 552 to move in a distal direction and engage with the gas/water valve 140. The user may actuate the second plunger 541 in a similar manner. For example, the user may actuate the second plunger 541 by pressing the second plunger 541 in a distal direction toward the second barrel 542, and the second plunger 541 forces a fluid (e.g., gas, water, saline, or the like) through the second tip 543 and the lumen of the second tube 522 into the second fluid chamber 553. The fluid creates pressure within the second fluid chamber 553, and the pressure then forces the second actuator button 554 to move in a distal direction and engage with the suction valve 145.

The actuator box 510 may be coupled to the central control section 115 of the endoscope 100, and may be include one or more actuator buttons (e.g., the first actuator button 552, the second actuator button 554). The actuator box 510 may be positioned over the one or more valves (e.g., the gas/water valve 140, the suction valve 145). In some cases, the actuator box 510 may be coupled to the central control section 115 via a fastener, such as, for example, a hook and loop fastener, a strap having a strap adjuster, a magnetic fastener, or any other fastener as desired.

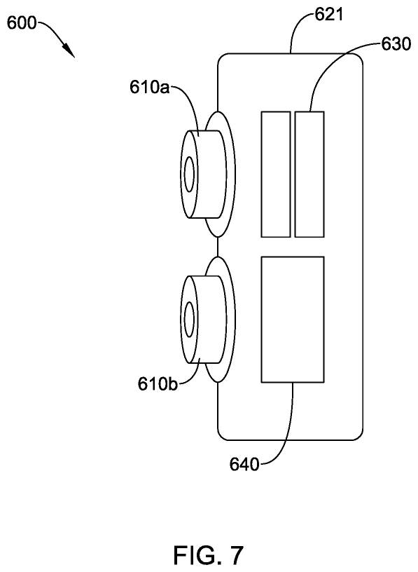

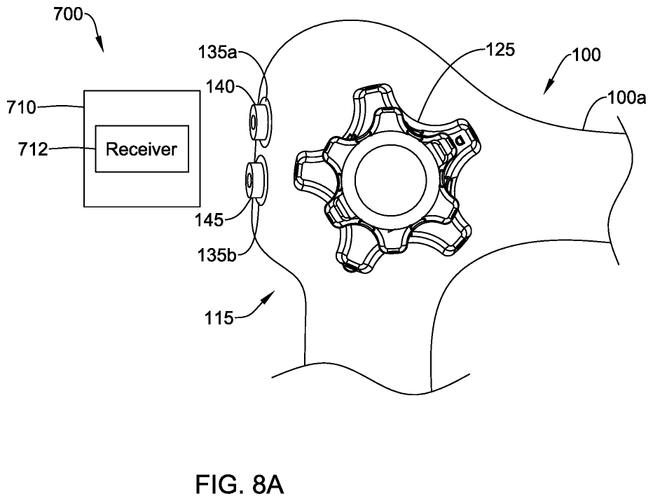

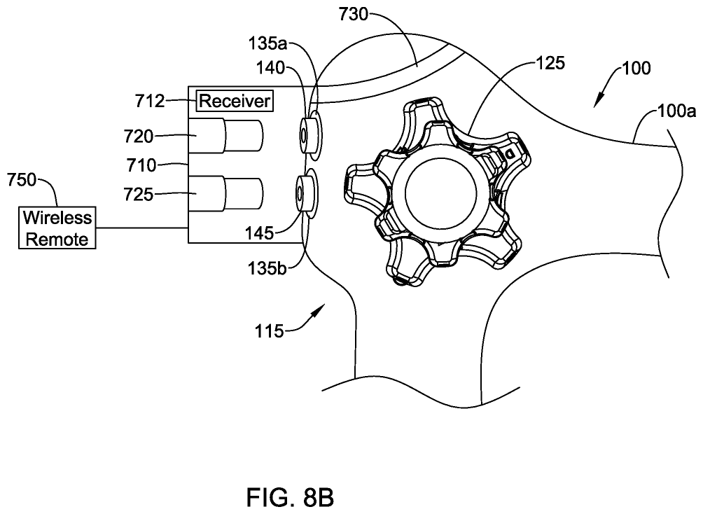

FIG. 7 illustrate exemplary remote user interfaces 600, and FIGS. 8A to 8B illustrate an actuator box 710 coupled to the central control section 115 of the endoscope 100. Turning to FIG. 7A, the remote user interface 600 may be a wireless remote configured to wirelessly communicate with the actuator box 710. The remote user interface 600 may include a housing 620, and a first button 610a and a second button 610b may be positioned on an outer surface 621 of the housing 620. The first button 610a and the second button 610b may be, for example, virtual buttons, physical buttons, or any other suitable type of button.

Further, with some embodiments, the remote user interface 600 may not include “buttons” are used in the conventional sense. For example, the remote user interface 600 may include a microphone, processor, and memory comprising instructions that when executed by the processor cause the processor to process audible tones, messages, words, or queues received by the microphone and identify one or more of these audible tones, messages, words, or queues as indicative of a press of the first button 610a and/or the second button 610b. As another example, the remote user interface 600 may be configured to be coupled to a robotic surgical device where the first button 610a and the second button 610b can be actuated (e.g., physically, electronically, etc.) by the robotic surgical device.

The remote user interface 600 may further include a power source 630, which may include, for example, one or more batteries. In some cases, the remote user interface 600 may include a transmitter 640 for sending information to a receiver 715, which may be positioned within the actuator box 710. In examples in which the remote user interface 600 is connected and communicates with the actuator box 710 over a wireless network, the transmitter 640 and the receiver 712 may be a wireless transmitter 640 and receiver 712.

The actuator box 710 may include a first actuator 720 configured to engage with the gas/water valve 140, and a second actuator 725 configured to engage with the suction valve 145. In some cases, when a user actuates the first button 610a and/or the second button 610b (e.g., touches, presses, or the like), the transmitter 640 sends a signal to the receiver 712 within the actuator box 710. In examples in which the user actuates the first button 610a, a signal is sent to the receiver 712 to actuate the first actuator 720 to move in a distal direction and engage with the gas/water valve 140. In examples in which the user actuates the second button 610b, a signal is sent to the receiver 712 to actuate the second actuator 725 to move in a distal direction and engage with the suction valve 145. In some cases, the actuator box 710 may be coupled to the central control section 115 via a fastener 730. The fastener 730 may be, for example, a hook and loop fastener, a strap having a strap adjuster, a magnetic fastener, or any other fastener as desired.

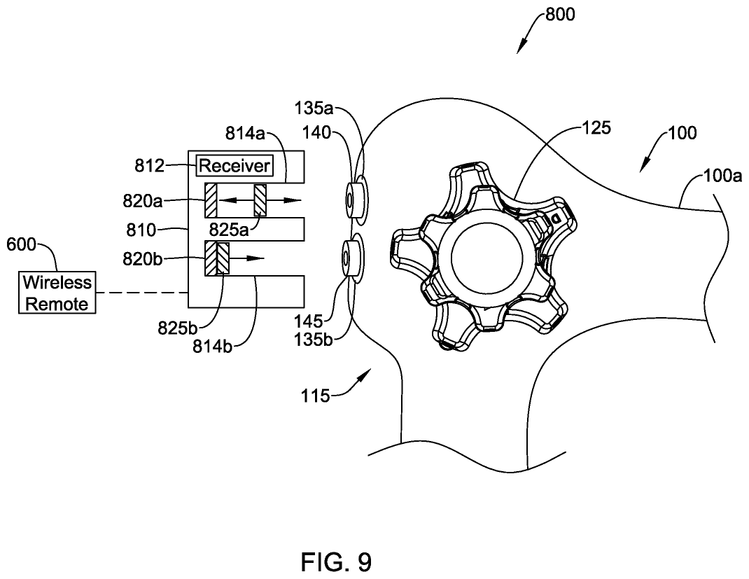

FIG. 9 illustrates an exemplary actuator box 810 coupled to the central control section 115 of the endoscope 100. The actuator box 810 may include a receiver 812, and may be configured to wirelessly communicate with the remote user interface 600 (e.g., a wireless remote). The actuator box 810 may be coupled to the central control section 115 via a fastener (not explicitly shown). The fastener may be, for example, a hook and loop fastener, a strap having a strap adjuster, a magnetic fastener, or any other fastener as desired.

The actuator box 810 may include one or more fixed magnets and one or more floating magnets. The one or more fixed magnets may be positioned within one or more channels 814a, 814b that may include a size and shape configured to fit the one or more fixed magnets and the one or more floating magnets. The one or more fixed magnets may be fixed within the one or more channels 814a, 814b via an adhesive attachment. The one or more floating magnets may not be fixed within the channel, but rather, may be free to move distally and/or proximally within the one or more channels 814a, 814b.

In some cases, the actuator box 810 may include a first fixed magnet 820a, a second fixed magnet 820b, a first floating magnet 825a, and a second floating magnet 825b. The remote user interface 600 may be configured to control a polarity of the first floating magnet 825a and the second floating magnet 825b such that the first floating magnet 825a and the second floating magnet 825b each include a first polarity and a second polarity. In some cases, the first polarity of the first floating magnet 825a may bias the first floating magnet 825a to a first position in which the first floating magnet 825a is attracted to the first fixed magnet 820a, and the first polarity of the second floating magnet 825b may bias the second floating magnet 825b to a first position in which the second floating magnet 825b is attracted to the second fixed magnet 820b, as illustrated by the second floating magnet 825b and the second fixed magnet 820b. In some cases, the second polarity of the first floating magnet 825a may bias the first floating magnet 825a to a second position in which the first floating magnet 825a is repelled from the first fixed magnet 820a, and the second polarity of the second floating magnet 825b may bias the second floating magnet 825b to a second position in which the second floating magnet 825b is repelled from the second fixed magnet 820b, as illustrated by the first floating magnet 825a and the first fixed magnet 820a.

When the first floating magnet 825a is in the second position (e.g., the first floating magnet 825a is repelled from the first fixed magnet 820a), the first floating magnet 825a may engage with the gas/water valve 140 to control a flow of fluid through the elongated shaft 100a of the endoscope 100. In some cases, when the second floating magnet 825b is in the second position (e.g., the second floating magnet 825b is repelled from the second fixed magnet 820b), the second floating magnet 825b may engage with the suction valve 145 to control a suction through the elongated shaft 100a of the endoscope 100.

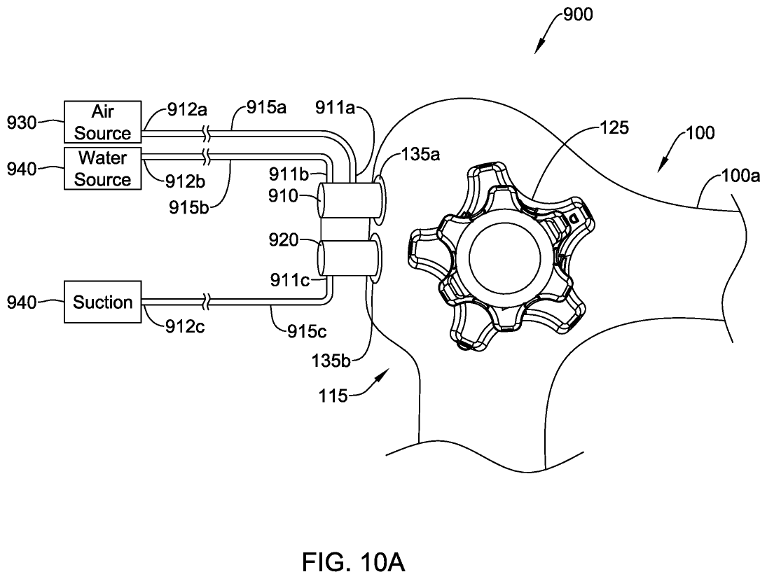

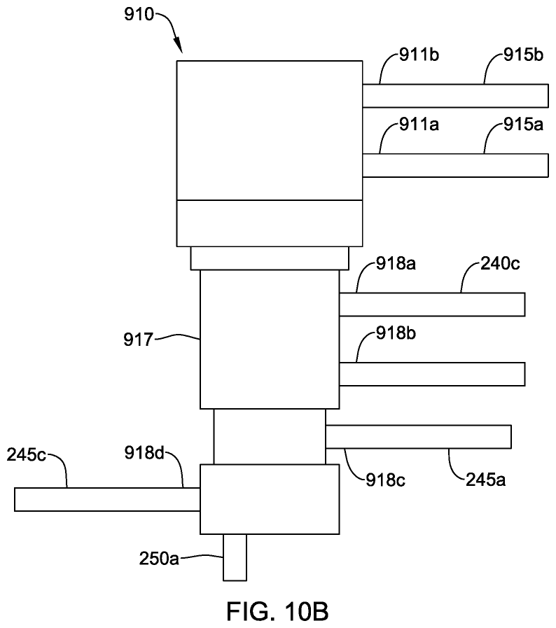

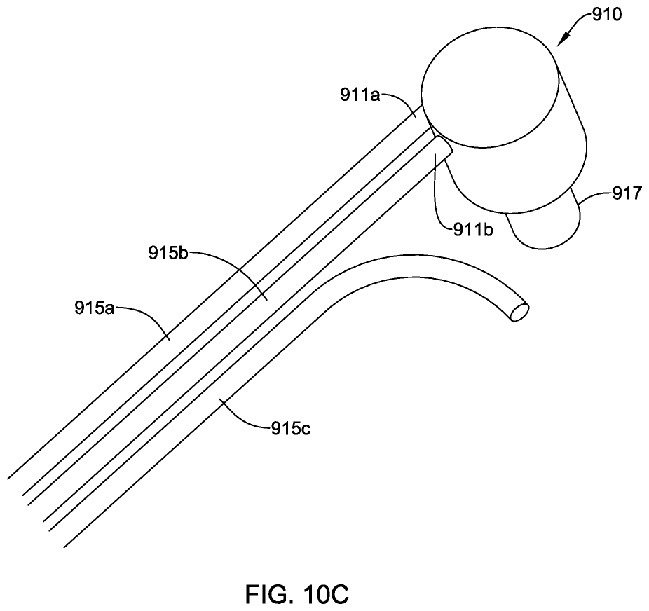

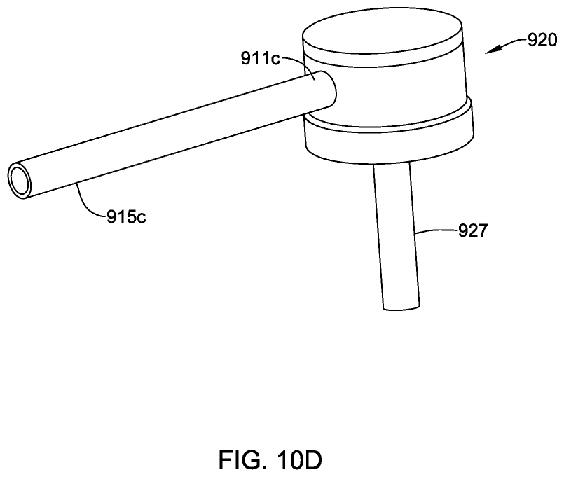

FIGS. 10A to 10D illustrate an exemplary first actuator button 910 and a second actuator button 920 coupled to the endoscope 100. As shown in FIGS. 10A to 10C, the first actuator button 910 may include a first fluid port 911a and a second fluid port 911b which may be configured to engage with a water supply tubing 915b and a gas supply tubing 915a, respectively. A proximal end 912b of the water supply tubing 915b may be coupled to a water source 940 (e.g., a water bottle), which may provide fluid for a lens wash. A proximal end 912a of the gas supply tubing 915a may be coupled to an air source, which may provide air for insufflation. The first actuator button 910 may be configured to engage with the first valve well 135a of the endoscope 100, and may include a first shaft 917, as shown in FIGS. 10B to 10C. The first shaft 917 may include one or more ports configured to engage with one or more fluid lines within the endoscope 100. For example, a first port 918a may be configured to engage with a gas supply tubing 240c configured to supply gas through the elongated shaft 100a of the endoscope 100. The second port 918b may be closed (e.g., blocked) from receiving a gas supply line as the gas supply tubing 915a is coupled to the first fluid port 911a. A third port 918c may be configured to engage with a lens wash supply line 245a configured to supply fluid through the elongated shaft 100a of the endoscope 100. A fourth port 918d may be closed (e.g., blocked) from receiving a lens wash tubing, as the water supply tubing 915b is coupled to the second fluid port 911b. A user may actuate the first actuator button 910 in a distal direction may advance the first shaft 917 a predetermined distance, such that each predetermined distance may expose the one or more ports as desired.

The second actuator button 920 may include a third fluid port 911c which may be configured to be coupled to a suction tubing 915c. A proximal end 912c of the suction tubing 915c may be configured to be coupled to a suction source to provide suction through the elongated shaft 100a of the endoscope 100. As shown in FIG. 10D, the second actuator button 920 may include a second shaft 927 that may be configured to engage with the second valve well 135b, and may be configured to provide a suction therethrough upon actuation.

The first actuator button 910 and the second actuator button 920 may replace the gas/water valve 140 and the suction valve 145, respectively, and may bypass all input lines (e.g., water supply tubing, gas supply tubing, suction tubing) that would otherwise be coupled to the endoscope 100 directly. Rather, the input lines may be coupled to the first actuator button 910 and the second actuator button 920. This may create a more streamlined process in which the tubing can be confined to one area, creating a less congested working area for a physician.

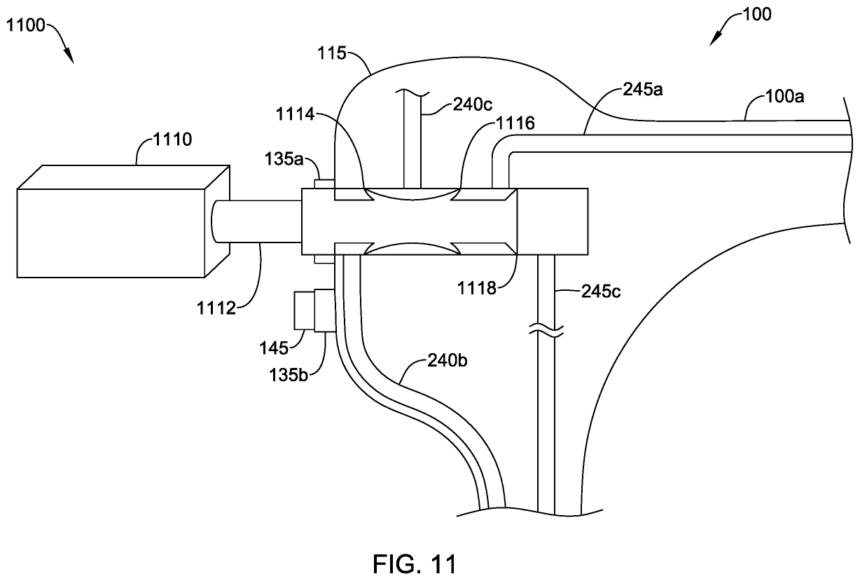

FIG. 11 illustrates a side view of an exemplary actuator button 1100. The actuator button 1100 may be a linear actuator button, and may be configured to be coupled to the central control section 115 of the endoscope via a valve well (e.g., valve well 135a, 135b). As shown in FIG. 11, the actuator button 1100 may be configured to engage with the first valve well 135a. The actuator button 1100 may be configured to engage with the first valve well 135a via a snap fit, a friction fit, or any other suitable type of connection. While it is not explicitly shown, the actuator button 1100 may also be configured to engage with the second valve well 135b.

The actuator button 1100 may include a body 1110 that may be configured to be depressed, or actuated in a distal direction. The actuator button 1100 may further include a shaft 1112 that maybe configured to fit within the first valve well 135a, and may include a first ring 1114, a second ring 1116, and a third ring 1118. The rings 1114, 1116, 1118 may be configured to provide a seal against an inside wall of the central control section 115 of the endoscope 100. The shaft 1112 may be configured to line up with corresponding wells within the endoscope 100. For example, the lens wash supply line 145a, the lens wash tubing 245c, the gas supply line 240a, and the gas supply tubing 240c. The actuator button 1100 may be actuated in a distal direction which may advance the shaft 1112 distally, thereby enabling the corresponding capability.

The endoscope 100 and portions thereof may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the endoscope 100 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of loading tool 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of loading tool 10 to achieve the same result.

In some embodiments, a degree of Magnetic Resonance Imaging (MRI) compatibility is imparted into the endoscope 100. For example, the endoscope 100, or portions thereof, may be made of a material that does not substantially distort the image and create substantial artifacts (i.e., gaps in the image). Certain ferromagnetic materials, for example, may not be suitable because they may create artifacts in an MRI image. The endoscope 100 or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An endoscope, comprising:
a handle including a central control section;
one or more valves coupled to the central control section;
an elongate shaft extending distally from the handle to a distal tip, the elongate shaft configured to be inserted into a body cavity; and
a remote user interface operatively coupled to the central control section, the remote user interface configured to operate the one or more valves to control a flow of fluid through the elongate shaft.

2. The endoscope of claim 1, wherein the remote user interface includes one or more buttons configured to be actuated to operate the one or more valves.

3. The endoscope of claim 2, wherein the one or more buttons are attached to one or more flexible tethers, the one or more flexible tethers configured to engage with the one or more valves.

4. The endoscope of claim 1, wherein the remote user interface includes one or more flexible tethers coupled to a foot pedal, the one or more flexible tethers configured to engage with the one or more valves.

5. The endoscope of claim 1, wherein the remote user interface includes an actuator box coupled to the central control section of the handle, the actuator box positioned over the one or more valves.

6. The endoscope of claim 5, wherein the remote user interface further includes a wireless remote configured to wirelessly communicate with a receiver positioned within the actuator box.

7. The endoscope of claim 6, wherein the actuator box includes one or more fixed magnets and one or more floating magnets, wherein the wireless remote is configured to control a polarity of the one or more fixed magnets such that the one or more fixed magnets include a first polarity in which the one or more fixed magnets attract the one or more floating magnets to a first position, and the one or more fixed magnets include a second polarity in which the one or more fixed magnets repel the one or more floating magnets to a second position, the one or more floating magnets configured to engage with the one or more valves when in the second position.

8. The endoscope of claim 6, wherein the actuator box includes one or more actuator buttons configured to engage with the one or more valves, wherein the wireless remote is configured to control the one or more actuator buttons.

9. The endoscope of claim 1, wherein the remote user interface includes one or more flexible tethers coupled to one or more syringes, the one or more flexible tethers configured to engage with the one or more valves and the one or more syringes configured to advance a fluid distally to control the flow of fluid through the elongate shaft.

10. An endoscope, comprising:
a handle including a central control section;
one or more valves coupled to the central control section, the one or more valves including one or more actuator buttons;
an elongate shaft extending distally from the handle to a distal tip, the elongate shaft configured to be inserted into a body cavity; and
a remote user interface operatively coupled to the central control section, the remote user interface configured to operate the one or more valves to control a flow of fluid through the elongate shaft;
wherein the remote user interface includes one or more buttons configured to be actuated to operate the one or more actuator buttons of the one or more valves.

11. The endoscope of claim 10, wherein the one or more actuator buttons are removed from the one or more valves, and replaced with one or more tethers, the one or more tethers coupled to the remote user interface.

12. The endoscope of claim 10, wherein the remote user interface includes an actuator box coupled to the central control section of the handle, the actuator box positioned over the one or more actuator buttons of the one or more valves.

13. The endoscope of claim 12, wherein the remote user interface further includes a wireless remote configured to wirelessly communicate with a receiver positioned within the actuator box.

14. The endoscope of claim 10, wherein the remote user interface further includes a wireless remote configured to wirelessly communicate with a receiver positioned at the control section of the handle.

15. The endoscope of claim 13, wherein the actuator box includes one or more fixed magnets and one or more floating magnets, wherein the wireless remote is configured to control a polarity of the one or more fixed magnets such that the one or more fixed magnets include a first polarity in which the one or more fixed magnets attract the one or more floating magnets to a first position, and the one or more fixed magnets include a second polarity in which the one or more fixed magnets repel the one or more floating magnets to a second position, the one or more floating magnets configured to engage with the one or more actuator buttons when in the second position.

16. An endoscope, comprising:
a handle including a central control section;
one or more actuator buttons coupled to the central control section;
a receiver positioned adjacent the control section of the handle;
an elongate shaft extending distally from the handle to a distal tip, the elongate shaft configured to be inserted into a body cavity; and
a remote user interface operatively coupled to the central control section, the remote user interface configured to operate the one or more actuator buttons to control a flow of fluid through the elongate shaft;
wherein the remote user interface includes a wireless remote configured to wirelessly communicate with the receiver.

17. The endoscope of claim 16, wherein the receiver is positioned within an actuator box, the actuator box coupled to the handle.

18. The endoscope of claim 17, wherein the actuator box includes one or more buttons configured to engage with the one or more actuator buttons.

19. The endoscope of claim 16, wherein the remote user interface includes one or more sensors configured to communicate with the receiver.

20. The endoscope of claim 17, wherein the actuator box includes one or more fixed magnets and one or more floating magnets, wherein the wireless remote is configured to control a polarity of the one or more fixed magnets such that the one or more fixed magnets include a first polarity in which the one or more fixed magnets attract the one or more floating magnets to a first position, and the one or more fixed magnets include a second polarity in which the one or more fixed magnets repel the one or more floating magnets to a second position, the one or more floating magnets configured to engage with the one or more actuator buttons when in the second position.
